(19) United States
(12) Patent Application Publication
(10) Pub. No.: US 2021/0330364 A1
(43) Pub. Date: Oct. 28, 2021

(54) SPREADING AND CONTRACTING TOOL FOR KNEE OSTEOTOMIES

(71) Applicant: LABORATOIRES BODYCAD INC., Québec (CA)

(72) Inventors: Jean Robichaud, St- Aubert (CA); Hugo Robichaud, Quebec (CA); Geoffroy Rivet-Sabourin, Stoneham (CA)

(21) Appl. No.: 16/610,468

(22) PCT Filed: Aug. 22, 2019
(86) PCT No.: PCT/CA2019/051156

§ 371 Date: Nov. 1, 2019

Related U.S. Application Data
(60) Provisional application No. 62/722,403, filed on Aug. 24, 2018.
(60) Provisional application No. 62/722,451, filed on Aug. 24, 2018.

(51) Int. Cl. A61B 17/80; A61B 17/88; A61B 17/17; A61B 90/00

(52) U.S. Cl. CPC A61B 17/8095; A61B 17/8866; A61B 2090/067; A61B 90/06; A61B 17/1764

(57) ABSTRACT

A tool for spreading and contracting a bone along a cut formed therein as part of a knee osteotomy procedure is provided. The spreading tool includes an upper arm and a lower arm respectively extending along a length between an effort end and a load end, the arms being pivotally connected to one another via a hinge positioned between the effort and load ends; and an anchor interface proximate the load ends for anchoring the load ends of the arms relative to respective first and second fixed positions on the bone. The tool being operable, via rotation of the arms about the hinge, between a closed configuration in which the load ends of the upper and lower arms are proximate one another and an open configuration in which the load ends of the upper and lower arms are spaced apart from one another.

### TECHNICAL FIELD

The technical field generally relates to tools used in knee osteotomy procedures, and more particularly in high tibial osteotomies.

### BACKGROUND

Knee osteotomies are orthopedic procedures which aim to correct the alignment of knee joints to adjust pressure distribution. A high tibial osteotomy is a type of knee osteotomy which involves correcting the alignment of a knee joint by reconfiguring the mechanical axis of the tibia. Depending on the required correction angle, the high tibial osteotomy can be an open wedge osteotomy or a closed wedge osteotomy. In an open wedge osteotomy, a planar cut is made in the tibia below the knee, and the tibia bone is opened along the planar cut to form a wedge-shaped opening with a specified angle. In a closed wedge osteotomy, a wedge of bone having a specified angle is removed from the tibia bone below the knee, and the tibia bone is closed along the wedge. After the bone is opened or closed, it is retained in place by installing a fixation plate. The opening or closing effectively adjusts the angle of the tibia relative to the femur, thereby reconfiguring how pressure between the tibia and the femur is distributed in the knee.

Existing tools and procedures are limited in the accuracy and precision with which the alignment of the knee can be corrected. There is therefore much room for improvement.

### SUMMARY

According to an aspect, a tool for spreading and/or contracting a bone along a cut formed therein as part of a knee osteotomy procedure is provided. The spreading tool includes: an upper arm and a lower arm respectively extending along a length between an effort end and a load end, the upper and lower arms being pivotally connected to one another via a hinge positioned between the effort and load ends; and an anchor interface proximate the load ends for respectively anchoring the load ends of the upper and lower arms relative to respective first and second fixed positions on the bone; the tool being operable, via rotation of the upper and/or lower arms about the hinge, between a closed configuration in which the load ends of the upper and lower arms are proximate one another and an open configuration in which the load ends of the upper and lower arms are spaced apart from one another.

According to an aspect, a tool for spreading and/or contracting a bone along a cut formed therein as part of a knee osteotomy procedure is provided. The tool includes: an upper arm and a lower arm respectively extending along a length between an effort end and a load end, the upper and lower arms being pivotally connected to one another via a hinge positioned between the effort and load ends; an anchor interface proximate the load ends for respectively anchoring the load ends of the upper and lower arms relative to respective first and second anchoring points on opposite sides of the cut in the bone; the tool being operable towards an open configuration in which a spreading force is applied across the first and second anchoring points via the load ends, and towards a closed configuration in which a contracting force is applied across the first and second anchoring points via the load ends.

### BRIEF DESCRIPTION OF THE DRAWINGS

FIG. 1A is a perspective view of a surgical guide secured to a patient's tibia bone, according to an embodiment; FIG. 1B is a top view of the surgical guide of FIG. 1A, showing drill holes formed through a cross section of the patient's tibia bone.

FIG. 2 is a perspective view of a predrilling module secured to an anchor module on the patient's tibia bone, according to an embodiment.

FIG. 3 is a perspective view of a spreading module, according to an embodiment; FIGS. 3A and 3B are side views showing operation of a spreading module respectively in a closed configuration and an open configuration.

### DETAILED DESCRIPTION

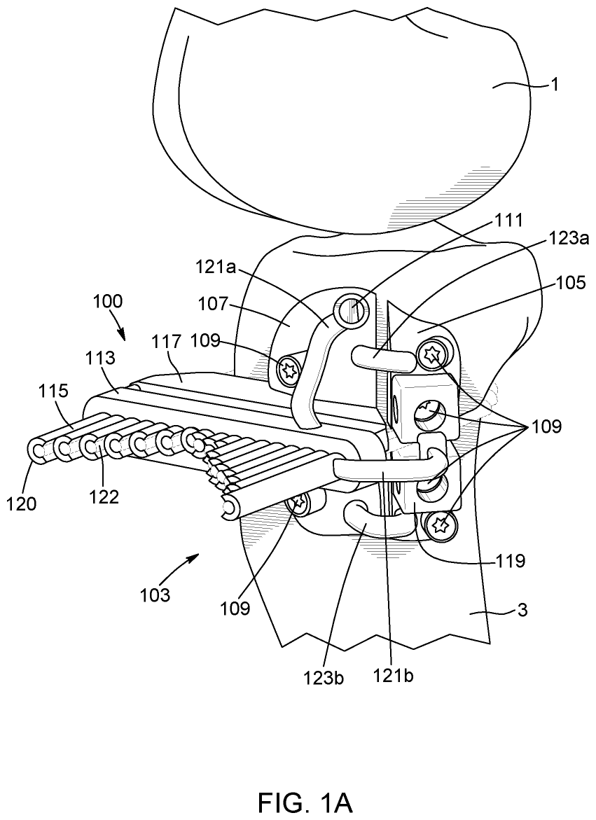

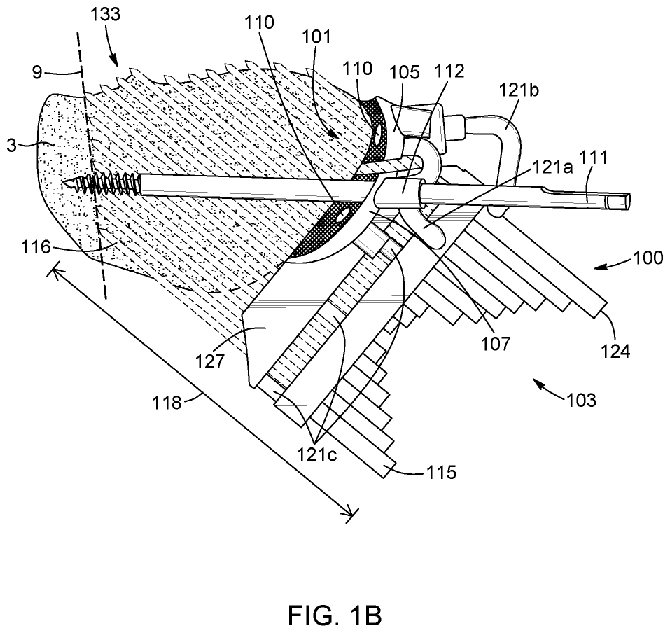

With reference to FIGS. 1A and 1B a surgical guide 100 is provided according to an embodiment. The surgical guide 100 is configured to be mounted to a patient's tibia bone 3 and includes a plurality of modules to guide various surgical tools used throughout the osteotomy procedure. The surgical guide 100 is patient-specific in that it is designed and manufactured according to the specific anatomy of a patient. In this fashion, the surgical guide 100 can be shaped and configured such that it can fit precisely on a predetermined position on the patient's bone 3 and be secured thereto to assure proper alignment of guides for various surgical tools. In the present embodiment, the surgical guide 100 has a body made from 3D printed plastic, although it is appreciated that other biocompatible materials compatible with other custom manufacturing methods are also possible.

The body of surgical guide 100 comprises a bone interface side 101 for facing the patient's bone 3, and an operative side 103 for facing away from the patient's bone 3. In the present embodiment, bone interface side 101 is configured to be positioned directly on the patient's bone, and comprises a surface having contours complementary is shape to the surface contours of a predetermined area of the patient's bone 3. In this configuration, bone interface side 101 can abut against the patient's bone, and key into a specific position thereon. In the present embodiment, bone interface side 101 comprises a solid surface, however it is appreciated that other configurations are possible. For example, the surface can be defined by an open lattice, and can comprise edges conforming to the contours of the patient's bone 3. Operative side 103 is provided opposite interface side 101 and includes a variety of components for interacting with surgical tools, as will be described in more detail hereinafter.

In the present embodiment, the body of surgical guide 100 is subdivided into two separable sections, including a lateral section 105 for securing relative to a lateral or medial surface of the patient's bone 3 and an anterior section 107 for securing relative to an anterior surface of the patient's bone 3. It is appreciated, however, that in other embodiments, more or fewer sections are possible to secure relative to different surfaces of the patient's bone 3 depending on surgical requirements. In the present embodiment, lateral section 105 and anterior section 107 are independently securable relative to the patient's bone 3. In this fashion, the lateral 105 or anterior 107 section can be removed from the patient's bone 3 when no longer needed, while the other section can remain secured in place. In the present embodiment, lateral 105 and anterior 107 sections are secured directly to the patient's bone, however it is appreciated that in some embodiments, only one of the lateral 105 and anterior 107 need be affixed directly to the bone. For example, lateral section 105 can be affixed directly to the bone 3, whereas anterior section 107 can be removably attached to lateral section 105 such that it is secured relative the patient's bone 3 without being directly affixed thereto.

In the present embodiment, lateral 105 and anterior 107 sections comprise bone-conforming plates secured to the patient's bone 3 via fasteners. The fasteners comprise surgical screws 109 although it is appreciated that other types of fastening mechanisms are also possible. The screws 109 engage in the patient's bone 3 through canals 110 opening on the bone interface 101 and operative 103 sides of the surgical guide 100. The canals 110 comprise sidewalls extending along a length for guiding insertion of screws 109 through canals 110 at a specified angle and depth. In this fashion, screws 109 drilled into the patient's bone 3 through canals 110 can be guided into a predetermined position, orientation and depth such that they can secure patient-specific surgical guide 100 to the patient's bone 3 in an optimal fashion, and such that the screws 109 will not interfere with tools used during subsequent steps during the osteotomy procedure. The sidewalls of canals 110 can further be configured to abut against a head of screw 109 to block the screw 109 from being inserted too deep into the patient's bone 3.

In the present embodiment, a plurality of canals 110 are provided for securing the surgical guide 100 to the patient's bone 3 via a plurality of screws 109 at strategic locations. It is appreciated, however, that in other embodiments, a different number of screws 109 and canals 110 can be provided, and that they can be positioned and oriented differently depending on the patient's specific anatomy and according to the planned procedure. Moreover, in the present embodiment, each of screws 109 is the same size, but it is appreciated that in other embodiments, different sized screws can be used to secure different parts of the surgical guide 100, and that the canals 110 can be sized and shaped accordingly. Finally, although the screws 109 are guided by canals 110 in the present embodiment, it is appreciated that other screw-guiding mechanisms are possible in other embodiments.

As mentioned above, lateral 105 and anterior 107 sections are separable from one another. In the present embodiment, lateral 105 and anterior 107 sections are generally disjointed from one another and are connected via connecting members. In other words, lateral 105 and anterior 107 sections are not directly fused together, and instead comprise separate spaced-apart sections removably secured to one another at a finite number of fixed points. In this configuration, each of lateral 105 and anterior 107 sections define two separate bone-contacting surfaces including two bone-conforming plates on bone interface side 101 of surgical guide 100. It is appreciated, however, that in other embodiments, lateral 105 and anterior 107 sections can together form a single coherent surface or plate for contacting the bone 3.

Connecting members 121, 123, can be provided to removably connect different sections of the surgical guide 100. In the present embodiment, the lateral 105 and anterior 107 sections are connected to one another at three fixed points via connecting members 121b, 123a and 123b. The connecting members 121b, 123a, 123b are stems comprising narrow strands of rigid material connected at a first end to the lateral section 105 and at a second end to the anterior section 107. The connecting members 121b, 123a, 123b are fused to lateral 105 and anterior 107 sections and/or are formed as integral parts thereof. In this fashion, lateral 105 and anterior 107 sections can be rigidly connected to one another and can be disconnected by respectively severing each of connecting members 121b, 123a, 123b. Connecting members 121, 123 are configured such that an intermediate portion thereof is spaced away from surgical guide 100 and/or the patient's bone 3, thereby allowing the connecting members 121, 123 to be readily severed using a severing tool (such as a saw or scissors, for example) while minimizing a risk of damaging surgical guide 100 or bone 3. In the present configuration, connecting members 121b, 123a, 123b loop away from the surgical guide 100 and comprise a rounded intermediate section spaced away from surgical guide 100. Although a particular configuration of connecting members 121, 123 has been shown, it is appreciated that other configurations are possible. In other embodiments, connecting members 121, 123 can have different shapes, and can include different connecting elements. For example, in some embodiments, instead of being fused and/or an integral part of lateral 105 and/or anterior 107 sections, connecting members 121, 123 can be separate pieces removably engageable in lateral 105 and/or anterior 107 sections. As can be further appreciated, in other embodiments, a different number of connecting members 121, 123 can be provided, and they can be positioned differently.

As mentioned above, the surgical guide 100 comprises a plurality of modules to guide various surgical tools used throughout the osteotomy procedure. Each module can perform a different function for assisting with various tasks throughout an osteotomy procedure. Some modules can form integral parts of the lateral 105 and/or anterior 107 sections secured directly to the patient's bone 3, whereas other modules can be independent elements which can be secured to relative to the patient's bone 3 by attaching to lateral 105 and/or anterior 107 sections. Although a particular set of modules will be described in detail hereinafter, it is appreciated that other modules and combinations thereof are possible depending on the requirements of the surgical procedure. Moreover, although some modules are described as performing particular functions, it is appreciated that some modules can perform two or more functions and/or have other advantages or uses not explicitly described herein, but that would be readily understood by a person of skill in the art upon reading the present disclosure.

Anchor Module

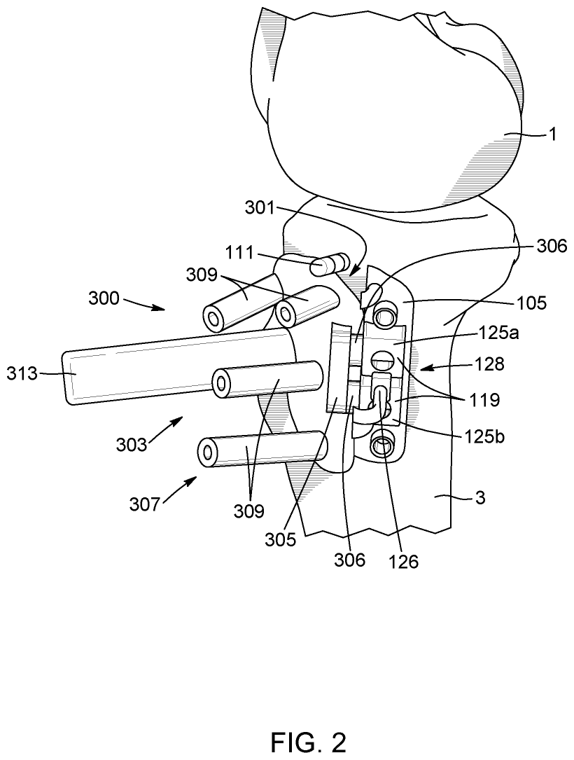

With reference now to FIG. 2, an anchor module 119 is provided to anchor removable modules relative to the patient's bone 3. In the present embodiment, anchor module 119 is provided in the lateral section 105 of the surgical guide 100, but it is appreciated that in other embodiments, anchor module 119 can be provided in a different section of guide 100. Moreover, in some embodiments, a plurality of anchor modules can be provided. The anchor module 119 is affixed directly to the patient's bone 3 via fasteners 109 and comprises a removable module interface 128 for interfacing with removable modules. The anchor module can thus act as a secure base to which other modules can be removably attached, allowing the removable modules to be properly aligned relative to the patient's bone 3 at relevant steps during the surgical procedure. In the present embodiment, the removable module interface 128 comprises apertures for receiving corresponding protrusions extending from a removable module, although it is appreciated that other removable connection interfaces are possible.

In the present embodiment, the anchor module 119 comprises two sections for providing two distinct anchoring points. More specifically, the anchor module 119 comprises a proximal section 125a positioned proximate the joint between the patient's femur 1 and tibia 3 bones, and a distal section 125b spaced further away from the joint between the femur 1 and tibia 3. The proximal 125a and distal 125b sections are separable from one another, allowing them to move independently while being secured to different sections of the patient's bone 3. In the present embodiment, proximal 125a and distal 125b sections are secured to one another via connecting member 126. The connecting member 126 can be severed to separate proximal 125a and distal 125b sections and allow them to move independently with different sections of bone. For example, in the present embodiment, proximal 125a and distal 125b sections are positioned on the patient's bone 3 on opposite sides of a planar cut formed therein as part of the surgical procedure. After the planar cut is formed, connecting member 126 can be severed to separate proximal 125a and distal 125b sections. The bone 3 can be opened along the planar cut, with the proximal 125a and distal 125b sections moving away from one another while being respectively connected to the bone 3 above and below the opening formed in the bone 3. In this fashion, the proximal section 125a can provide an anchoring point above or proximal the opening in the bone 3, while the distal section 125b provides an anchoring point below or distal the opening in the bone 3. It is appreciated that other positions and configurations of anchor module 119 and corresponding sections are possible, depending on the surgical procedure. It is further appreciated that the separable sections of anchor module 119 can be connected to one another via different removable connection mechanisms.

Spreader Module

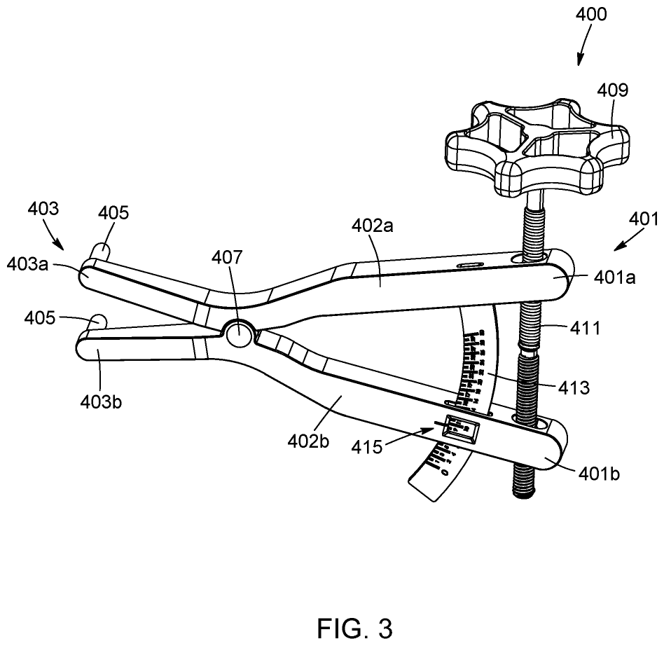

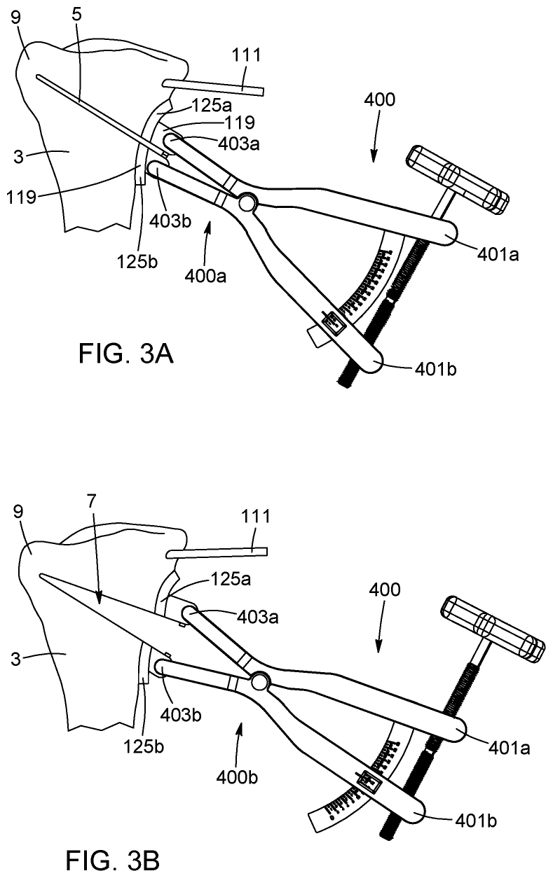

With reference now to FIGS. 3, 3A and 3B, a spreader module 400 (or spreading tool) to assist in spreading the patient's bone 3 is shown according to an embodiment. In the present embodiment, the spreader module 400 is configured to open the patient's bone 3 along a planar cut 5 formed therein. The planar cut 5 is opened at an angle about a hinge 9, thereby defining an open wedge 7 in the patient's bone. The spreader module 400 is configured to operate in cooperation with anchor module 119 secured to the patient's bone 3, but it is appreciated that other configurations are possible. As can be appreciated, the spreader module 400 can be a generic tool, and need not be custom made according to the patient. Instead, the surgical guide 100 can be designed to cooperate with generic spreader module 400. Accordingly, spreader module 400 can be made out of any rigid material, according to any manufacturing process. However, it is appreciated that in some embodiments, the spreader module 400 can be custom designed for the patient and to conform to a specific geometry of the guide 100. In such embodiments, the spreader module 400 can be made from materials suitable for custom manufacturing, for example from the same 3D printed plastic from which the surgical guide 100 and corresponding modules are made.

In the present, spreader module 400 comprises an upper arm 402a and a lower arm 402b pivotally connected to one another via a hinge 407. As can be appreciated, spreader module 400 is generally configured as a double lever, with an effort end 401 and a load end 403, and hinge 407 acting as a fulcrum therebetween. More specifically, as effort ends 401a, 401b of upper and lower arms 402a, 402b are moved towards one another, upper and lower arms 402a, 402b pivot about hinge 407 causing load ends 403a, 403b to move away from one another. In other words, a force applied at effort end 401 causing ends 401a and 401b to converge is transferred to load end 403, causing load ends 403a and 403b to separate. It is appreciated that other configurations of spreader module 400 are possible, so long as it permits a separating force to be applied to load ends 403a and 403b. For example, in some embodiments, the spreader module 400 can be configured such that a spreading of effort ends 401a, 401b transfers a spreading force to load ends 403a, 403b. In other embodiments, different types of spreading mechanisms are possible.

In some embodiments, the spreader module 400 can include an actuating assembly 408 operable to apply a force on effort end 401 for moving effort ends 401a, 401b towards and/or away from one another. In other words, the actuating assembly 408 is operable to pivot the arms 402a, 402b about hinge 407. In the present embodiment, force on effort end 401 is applied via a hand wheel 409. As wheel 409 is operated, screw mechanism 411 rotates and engages in threaded bores in effort ends 401a, 401b, thereby drawing effort ends 401a, 401b together or spreading them apart depending on the rotating direction of screw 411. As can be appreciated, in this configuration, a rotational force applied to wheel 409 is converted into a linear force which draws effort ends 401a, 401b together or spaces them apart. Moreover, the rotational force applied to and wheel 409 merely causes a change in spacing of effort ends 401a, 401b. A constant force does not need to be applied to wheel 409 to retain effort ends 401, 401b at a fixed spacing; instead, when no force is applied, the engagement of screw mechanism 411 retains arms 402 of spreader module 400 at their current angle, retaining effort ends 401a, 401b at a fixed spacing until force is applied to wheel 409. Spacing of effort ends 401a, 401b can thus be precisely controlled by hand, via small and/or measured rotational movements of hand wheel 409. It is appreciated, however, that a force controlling spacing of effort ends 401a, 401b can be applied via different mechanisms, and that such mechanisms need not necessarily be operated by hand. For example, in some embodiments, force can be applied via hydraulics or motors, and/or can be controlled electronically.

As mentioned above, spreader module 400 is configured to cooperate with anchor module 119 secured to the patient's bone 3. Spreading module 400 comprises an anchor interface 405 at load end 403 for interfacing with anchor 119 and transferring spreading force thereto. More specifically, in the present embodiment, the anchor interface 405 comprises protrusions or pins sized and shaped to engage in corresponding apertures in anchor module 119. A protrusion or pin at load end 403a of upper arm 402a is positioned to engage with proximal section 125a of anchor module 119, whereas a protrusion or pin at load end 403b or lower arm 402b is positioned to engage with distal section 125b of anchor module. In this configuration, arms 402a, 402b of spreader module independently engage in the distinct anchoring points 125a, 125b, allowing arms 402a, 402b to apply a spreading force thereon in opposite directions, and move anchoring points 125a, 125b away from one another.

In the present embodiment, the protrusions or pins extend from arms 402a, 402b substantially perpendicular therefrom, and along an axis substantially parallel to the pivot axis of hinge 407. As can be appreciated, in this configuration, spreader module 400 can engage with anchor 119 by sliding protrusions or pins of anchor interface 405 laterally into the corresponding apertures of anchor 119. A vertical spreading force can be subsequently applied to arms 402a, 402b without causing interface 405 to disengage. In the same manner, spreader module 400 can be easily disengaged from anchor 119 by sliding the protrusions or pins out along the lateral direction. As can be further appreciated, in this configuration, spreader module 400 can engage with anchor module 119 and operate along the lateral section of the patient's bone 3, leaving anterior section of the bone 3 clear so as to not interfere with subsequent steps in the surgical procedure. Apertures in anchor module 119 open on both anterior and lateral sides thereof, allowing the spreader module 400 to engage on either the anterior or lateral side of anchor module 119 depending on the requirements of the surgical procedure. It is appreciated, however, that in other embodiments, spreader module 400 can engage on other sides of anchor module 119, such as on its front side, and/or on top/bottom sides.

In the present embodiment, pins or protrusions of anchor interface 405 are substantially cylindrical and engage in substantially circular apertures in anchor module 119. As can be appreciated, in this configuration, pins or protrusions can rotate freely inside apertures of anchor module 119, allowing relative angular displacement of ends 403a, 403b relative to anchoring points 125a, 125b while engaged therein. It is appreciated, that in other embodiments, anchor interface 405 and/or anchor module 119 can comprise different engagement mechanisms. For example, in some embodiments, anchor interface 405 can be secured to anchor module 119 via fasteners. In some embodiments, ends 403a, 403b can key into anchoring points 125a, 125b at specific relative orientations, and/or pins or protrusions can be pivotally secured to ends 403a, 403b of arms 402a, 402b.

Spreader module 400 is operable to move between a closed configuration 400a and an opened configuration 400b. In the closed configuration 400a, anchor interface 405 on load ends 403a, 403b are substantially proximate one another and aligned with anchoring points 125a, 125b prior to spreading the patient's bone 3. In the opened configuration 400b, anchor interface 405 on load ends 403a, 403b are spaced apart from one another, and load end 403a, 403b are angled relative to one another at an opening angle. In the present embodiment, a gauge 413 is provided to indicate the magnitude of opening angle. The gauge 413 comprises a scale affixed to upper arm 402a, and movable through a corresponding aperture in lower arm 402b. A window 415 in lower arm 402b provides a visual indicator for reading the scale. It is appreciated, however, that other gauge mechanisms are possible to indicate the magnitude of opening angle. In the present embodiment, gauge 413 is calibrated such that scale is zeroed when the spreader module 400 is in the closed configuration 400a. The opening angle indicated by gauge 413 can thus provide an accurate and precise indication of the opening angle of spreader module 400. In some embodiments, the gauge 413 can be further calibrated such that it corresponds to the opening angle about hinge 9 in patient's bone 3. In this configuration, the gauge can provide a precise and accurate indication of opening angle of the open wedge 7 formed in the patient's bone, as the bone is opened along cut 5 using spreader module 400.

Although the module 400 is referred to herein as a “spreader” module or tool, it is appreciated that it can be used not only to spread the patient's bone 3, but also to contract the patient's bone 3, for example as part of a closed-wedge osteotomy. In such procedures, the spreader module 400 can be operated to draw anchoring points 125a, 125b closer together, for example to close an open wedge 7 cut into the patient's bone 3. More particularly, spreader module 400 can engage with anchoring points 125a, 125b while in the opened configuration 400b, with the anchoring points 125a, 125b being positioned on opposite sides of an open wedge 7. The spreader module 400 can be subsequently operated towards the closed configuration 400a by turning hand wheel 409, thereby drawing anchoring points 125a, 125b together and closing the wedge 7.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative and non-limiting and it will be understood by persons skilled in the art that other variants and modifications may be made without departing from the scope of the invention as defined in the claims appended hereto.

1. A tool for spreading and/or contracting a bone along a cut formed therein as part of a knee osteotomy procedure, the spreading tool comprising:
an upper arm and a lower arm respectively extending along a length between an effort end and a load end, the upper and lower arms being pivotally connected to one another via a hinge positioned between the effort and load ends; and
an anchor interface proximate the load ends for respectively anchoring the load ends of the upper and lower arms relative to respective first and second fixed positions on the bone;
the tool being operable, via rotation of the upper and/or lower arms about the hinge, between a closed configuration in which the load ends of the upper and lower arms are proximate one another and an open configuration in which the load ends of the upper and lower arms are spaced apart from one another.

2. The tool according to claim 1, wherein the upper and lower arms extend opposite one another between the effort and load ends.

3. The tool according to claim 1, wherein the upper and lower arms are substantially arcuated, and extend away from one another between the hinge and the load ends and/or between the hinge and the effort ends.

4. The tool according to claim 1, wherein the anchor interface is adapted to engage an anchor module secured on a surface of the bone.

5. The tool according to claim 4, wherein the anchor interface comprises protrusions extending from the load ends of the upper and lower arms, said protrusions being adapted to respectively engage in first and second anchoring points of the anchor module positioned on the bone on opposite sides of the cut.

6. The tool according to claim 5, wherein each protrusion and corresponding arm are made as a one-piece unit.

7. The tool according to claim 5, wherein the protrusions extend substantially perpendicularly from the arms.

8. The tool according to claim 5, wherein the protrusions are cylindrical and have respective cylindrical axes.

9. The tool according to claim 8, wherein the protrusions engage respective anchoring points via a sliding movement in a direction substantially parallel to the cylindrical axes.

10. The tool according to claim 9, wherein the protrusions are configured to toolessly engage the anchoring points of the anchor module.

11. The tool according to claim 8, wherein the protrusions are adapted to rotate about their respective cylindrical axis relative to the anchoring points in which they are respectively engaged.

12. The tool according to claim 1, further comprising an actuating assembly operatively connected to the effort ends of the upper and lower arms, operable to pivot the upper and/or lower arms about the hinge.

13. The tool according to claim 12, wherein the upper and lower arms respectively have a threaded bore extending therethrough proximate the effort ends, and wherein the actuating assembly comprises a screw mechanism extending through the threaded bores and being adapted to pivot the arms about the hinge upon rotation of the screw mechanism.

14. The tool according to claim 13, wherein the screw mechanism is adapted to retain the spacing of effort ends when the actuating assembly is not operated.

15. The tool according to claim 13, wherein the actuating assembly further comprises a hand wheel connected to the screw mechanism for facilitating rotation of the screw mechanism by hand.

16. The tool according to claim 1, further comprising a gauge extending between the upper and lower arms for indicating a magnitude of an opening angle defined between the load ends.

17. The tool according to claim 16, wherein the gauge comprises a scale connected to the upper arm, and movable through an aperture provided in the lower arm.

18. The tool according to claim 17, wherein the lower arm comprises a window communicating with the aperture to allow reading the scale through the window.

19. The tool according to claim 1, wherein the upper and lower arms are made from a rigid material.

20. The tool according to claim 1, wherein the upper and lower arms are made from 3D-printable material.

21-39. (canceled)
